(19) United States
(12) Patent Application Publication
(10) Pub. No.: US 2014/0335021 A1
(43) Pub. Date: Nov. 13, 2014

(54) Ocular Neostigmine Drops for Diagnosing Myasthenia Gravis

(71) Applicants: Mustafa A.M. Salih, Riyadh (SA); Mohamed A.M. Salih, Omdurman (SD); Ali A. Mustafa, Riyadh (SA); Thomas M. Bosley, Riyadh (SA)

(72) Inventors: Mustafa A.M. Salih, Riyadh (SA); Mohamed A.M. Salih, Omdurman (SD); Ali A. Mustafa, Riyadh (SA); Thomas M. Bosley, Riyadh (SA)

(21) Appl. No.: 13/892,624

(22) Filed: May 13, 2013

(51) Int. Cl. A61K 49/00

(52) U.S. Cl. CPC A61K 49/0004
USPC 424/9.1

(57) ABSTRACT

Disclosed is a method for aiding in the diagnosis of myasthenia gravis. Neostigmine, for example one drop of standard intravenous neostigmine solution (2.5 mg/ml) is instilled into each symptomatic or ptotic eye of patients with suspected or probable myasthenia gravis. The patient is later evaluated for possible changes in ptosis. An observable increase of the palpebral fissure height of at least 2 mm, e.g., after 30-60 minutes after neostigmine instillation is considered to be diagnostic.

Myasthenia gravis (MG) is an acquired autoimmune disorder caused by autoantibodies directed against epitopes on or around the acetylcholine receptor (AChR) in the muscle membrane. The condition is characterized clinically by variable ptosis, ocular motility abnormalities, and weakness of voluntary muscles in other parts of the body. Nearly all patients present with extraocular muscles involvement, causing ptosis and diplopia, and in 20-40% of patients, weakness remains limited to these muscles (ocular MG). The diagnosis may be difficult to make on clinical grounds especially in juvenile cases when weakness is restricted to few muscles such as in ocular MG, and a variety of supportive diagnostic tests have been devised.

The best known of these is the Tensilon test, using intravenous edrophonium chloride, which is an acetylcholinesterase (AChE) inhibitor with very short action. Unfortunately, edrophonium causes cholinergic side effects that may include increased oral and bronchial secretions, abdominal cramps, sweating, bradycardia, heart block, hypotension, and red, watery, painful eyes. These are often uncomfortable for the patient, and although serious complications of bradycardia and syncope are rare the test is now commonly performed with cardiac monitoring and atropine available as an anti-cholinergic agent. Other relative contraindications for performance of edrophonium are cardiac dysrhythmias and bronchial asthma. A second quick bedside test is by applying an ice pack locally to a ptotic eyelid for 2-5 minutes and observing for any improvement of ptosis or eye movement deficit. The ice pack test is simple, inexpensive, and has a reported sensitivity of 93-97% and specificity of 97-98%. This adjunctive diagnostic test is particularly helpful if the edrophonium test is contraindicated or not available. However, the procedure is unlikely to be tolerated by children.

Other supportive tests require laboratory and electrophysiologic facilities. Serologic tests include assessing AChR antibodies which are found in 80% of adults with MG, 70% of peripubertal juvenile MG but only 50% of prepubertal MG. Antibodies to muscle-specific kinase (MuSK) are rare in juvenile MG. Electrodiagnosis of MG includes repetitive nerve stimulation [RNS] (looking for a decrement of greater than 10% of the fifth compared to the first-evoked compound muscle action potential [CMAP] in multiple muscles). However, the test is uncomfortable in children, is artifact prone and can be abnormal in disorders other than MG. The reported sensitivity of RNS ranges between 53% and 100%, but it is frequently negative in ocular MG. Single-fiber electromyography (SFEMG) requires a significant degree of cooperation and expertise and is not available in many hospitals. In young children, sedation may be required to facilitate both RNS and stimulated SFEMG. An additional simple, safe, sensitive, and specific diagnostic test for MG is, therefore, needed.

### SUMMARY

Provided herein is a method for aiding in the diagnosis of myasthenia gravis, the method comprises administering a solution of neostigmine into a ptotic (drooping) eye of a patient suspected of having myasthenia gravis. After a suitable time period for the composition to act on the eye, the eyelid is observed for an increase in palpebral fissure height, that is, an increase in the distance between the lower and upper eyelids. An increase of at least 2 mm, 30-60 minutes after administration of neostigmine is considered to be diagnostic of myasthenia gravis.

According to one non-limiting example, the method comprises the steps of installing one drop of standard intravenous neostigmine solution (2.5 mg/ml) into each symptomatic eye of patients with probable myasthenia gravis. In one embodiment, the eyes are documented before and after administration of neostigmine by photography in order to facilitate archival and objective determination of palpebral fissure height. After administration of the neostigmine, the eye lids are closed for at least 2-3 minutes in order to keep the drop inside the eye and allow for absorption. At 30-60 minutes, the patient is evaluated for possible changes in ptosis. An observable increase of the palpebral fissure height of at least 2 mm, 30 or 60 minutes after neostigmine administration is considered to be diagnostic.

### BRIEF DESCRIPTION OF THE DRAWINGS

FIG. 1: Mean pupil diameter measurements of the right and left eyes of 30 rabbits over time. One drop of neostigmine solution (2.5 mg/ml) was instilled in the right eye, whereas one drop of saline was instilled in the left (control) eye.

FIG. 2: Pupillary response to instillation of neostigmine (right eye) and saline (left eye). Images A-D=right eye; Images E-H=left eye. A=Pre neostigmine (6.0 mm); B=30 mins post neostigmine (2.0 mm); C=60 mins post neostigmine (1.5 mm); D=1 week after daily instillation of 1 drop of neostigmine, last drop 24 hours previously (6.0 mm); E=Pre saline (7.0 mm); F=30mins post saline drop (7.5 mm); G =60 mins post saline drop (7.0 mm); H=1 week after daily instillation of 1 drop of saline, last drop 24 hours previously (7.0 mm).

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. All references are fully incorporated by such reference herein, solely to the extent of their technical disclosure and only such that it is consistent with this disclosure.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, the term “subject” refers to members of the animal kingdom including but not limited to human beings (human subjects) that are treated using the methods and compositions described herein.

As used herein, “pharmaceutically-acceptable,” means acceptable for use in humans and animals. “Ophthalmically acceptable” refers to acceptable for use in the eye of humans or animals. “Excipients” include, without limitation, one or more suitable: vehicle(s), solvent(s), diluent(s), pH modifier(s), buffer(s), salt(s), colorant(s), rheology modifier(s), antifoaming agent(s), hydrogel(s), surfactant(s), emulsifier(s), preservative(s), phospholipid(s), fatty acid(s), mono-, di- and tri-glyceride(s) and derivatives thereof, wax(es), oil(s) and water. The choice of excipient depends on the dosage form in question. In the present instance, a solution (liquid or gel) that is acceptable for ophthalmic use, such as an intravenous dosage form, is administered. According to one non-limiting example, the solution is a solution suitable for intravenous use. In another, the solution is suitable for ocular use. Non-limiting examples of suitable excipients include: water, saline (e.g. 0.9% saline), phosphate-buffered saline (PBS). Other ingredients may be included in the solution, so long as they do not interfere with the function of the methods described herein. In any case, according to the methods described herein, the drug product is administered ocularly, that is to the eye. A large variety of liquid dosage forms are known in the pharmaceutical arts, and many of which may be appropriate for treatment using the methods and compositions described herein (see generally, Troy, D B, Editor, Remington: The Science and Practice of Pharmacy, 21st Edition, Lippincott, Williams & Wilkins (2005), chapters 42 (“Intravenous Admixtures”) and 43 (“Ophthalmic Preparations”)).

Neostigmine is a compound having the formula:

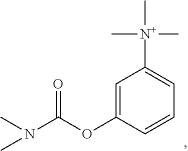

and is also known as 3-(trimethylamino)phenyl N,N-dimethylcarbamate (CAS No. 59-99-4), Prostigmin, Prostigmine, Eustigmine, Vagostigmine, Eustigmin, Intrastigmina, Juvastigmin, Neostigminum, Syntostigmine. It is a treatment for myasthenia gravis and is also used to reverse the effects of certain types of anesthesia on the muscles. The free base of neostigmine is provided above. Neostigmine drug products are typically available as a pharmaceutically acceptable salt. Neostigmine salts have a pharmaceutically acceptable conter-anion. Common suitable pharmaceutically acceptable acid addition salts include, without limitation, salts of inorganic acids such as hydrochloric acid, hydrobromic, phosphoric, metaphosphoric, nitric and sulfuric acid, and of organic acids such as, for example, acetic acid, benzenesulfonic, benzoic, citric, ethanesulfonic, fumaric, gluconic, glycolic, isethionic, lactic, lactobionic, maleic, malic, methanesulfonic, succinic, p-toluenesulfonic and tartaric acid. Particular examples of commercially-available neostigmine salts include chloride, bromide and methylsulphate salts. “Neostigmine” refers both to the neostigmine free base and pharmaceutically acceptable salts thereof.

Provided herein is a method for diagnosing myasthenia gravis. The method comprises administering a solution of neostigmine into a ptotic (drooping) eye of a patient suspected of having myasthenia gravis (e.g., probable myasthenia gravis). The neostigmine is administered in an amount effective to increase palpebral fissure height in a patient having myasthenia gravis. After a suitable time period for the composition to act on the eye, the eyelid is observed for an increase in palpebral fissure height, that is, an increase in the distance between the lower and upper eyelids. An increase of at least 2 mm, 30-60 minutes after administration of neostigmine is considered to be diagnostic of myasthenia gravis.

A ptotic eye is an eye with a drooping eyelid. This is in a patient suspected or probable of having myasthenia gravis. As indicated above, such signs or symptoms include ptosis, ocular motility abnormalities, such as diplopia, and weakness of voluntary muscles in other parts of the body. A suitable time period for the composition to act on the eye is a time period or window in which a given neostigmine composition can elicit its pharmacological effect on the eyelid muscles, yet still maintains that effect, can range from 5 minutes to two hours. Non-limiting examples of suitable end-points include 20, 30, 45, 60, 90 and 120 minutes. An amount effective to increase palpebral fissure height in a patient having myasthenia gravis can be any effective amount, for example 0.1 mg of neostigmine hydrochloride, as indicated below in the examples. Useful effective ranges can vary depending on the salt form and formulation, but would typically range from 0.01 mg to 1 mg, for example 0.05-0.5 mg or 0.1 mg. Useful effective ranges for any particular neostigmine formulation can be determined empirically using standard methodologies.

Also provided herein is a kit for diagnosing myasthenia gravis, including an ophthalmic drug product. The kit may comprise any acceptable packaging. The ophthalmic neostigmine drug product is sterile and according to one embodiment, is packaged in a controlled drop ophthalmic dispensing device (dispenser), for example a plastic reservoir container and a dropper tip, as are commonly and commercially available in the pharmaceutical arts for dispensing ophthalmic solutions in controlled droplet size. The container may be multi-use, containing enough of the neostigmine solution for many tests, or single use containing only a few drops worth of the solution for use and testing on a single patient. In one embodiment, the kit comprises more than one single-use dispensers. In one embodiment, the dropper tip comprises a break-away plastic closure to facilitate use and to preserve sterility until use. These types of dropper containers are broadly available in the relevant arts.

### EXAMPLES

### Materials and Methods

The present invention investigated the safety and efficacy of using intravenous (IV) neostigmine solution, available in all hospitals where anesthesia is performed, as an ocular drop for diagnostic purposes in the setting of patients with probable or definite autoimmune MG.

The study had two components: 1) experiments using 30 rabbits in which the standard IV neostigmine solution was instilled into one eye (using the other as control) to evaluate effectiveness (i.e., changes in the pupillary size of the experimental eye) and safety (i.e., does not cause significant irritation of the experimental eye or obvious systemic effects). 2) experiments in which the standard IV neostigmine solution was instilled into both eyes of patients with probable or definite symptomatic MG.

Both sets of experiments used the standard neostigmine (2.5 mg/ml) solution available in hospitals for anesthetic/surgical use for reversal of neuromuscular block. This solution is buffered and FDA-approved for use in humans. In both sets of experiments, one drop of this solution was used in an eye, limiting substantially the amount of drug that might be absorbed systemically.

### Rabbit Studies

Male New Zealand White rabbits (2.5-3.0 kg body weight) were used in all experiments. The animals were kept in a room of controlled room temperature (24.0+1.0° C.) and humidity and a fixed dark light cycle (light on at 7:00 am and off at 19:00 pm).

The sterile neostigmine solution (2.5 mg/ml) was instilled into the right eye (one drop), while the control eye (left eye) received one drop of sterile saline (0.9% sodium chloride). Assessment of pupillary size was done at 30 minute intervals following instillation of neostigmine. The left eye of each rabbit acted as control for the right one.

The amount of neostigmine methylsulphate was calculated to be 0.1 mg/drop. Therefore, each rabbit's eye was instilled by 0.1 mg of sterile neostigmine methylsulphate. The sterile dropper for instilling neostigmine was used only once and then discarded. To prevent the drop of neostigmine from leaking out of the rabbit's eye, the lower eye lid of the rabbits was held out for at least 2-3 minutes in order to keep the drop inside the eye and allow for better absorption of the drug through the cornea.

The pupil size of both the right and left eyes of each rabbit was measured at baseline in mms by using a plastic ruler. After instilling the neostigmine solution (right eye) or saline (left eye), the pupil size was measured again at 30 minute intervals following drug or saline instillation. One drop of neostimine was instilled daily for 7 days and signs of ocular inflammation were observed.

### Statistical Analysis

The mean values for the pupil sizes of the right eyes were recorded as well as those of the left eyes, separately. Paired t-test was used for comparison of data among the different groups on SPSS software. A probability value of <0.05 was considered to be significant.

### Human Studies

Patients with probable or definite MG were asked to sign informed consent approved by the appropriate Institutional Ethics Committees. Ptosis was recorded photographically, and pulse and blood pressure were also recorded prior to beginning the test. One drop of standard IV neostigmine solution (2.5 mg/ml) was instilled into each symptomatic eye of the patient. Fifteen minutes later, possible changes in ptosis were assessed. Pulse and blood pressure was recorded again and after another 15 minutes. At 30 minutes, the patient was evaluated also for possible changes in ptosis (documented by photography), obvious irritation of either eye and for report of discomfort.

### RESULTS

### Rabbit Studies

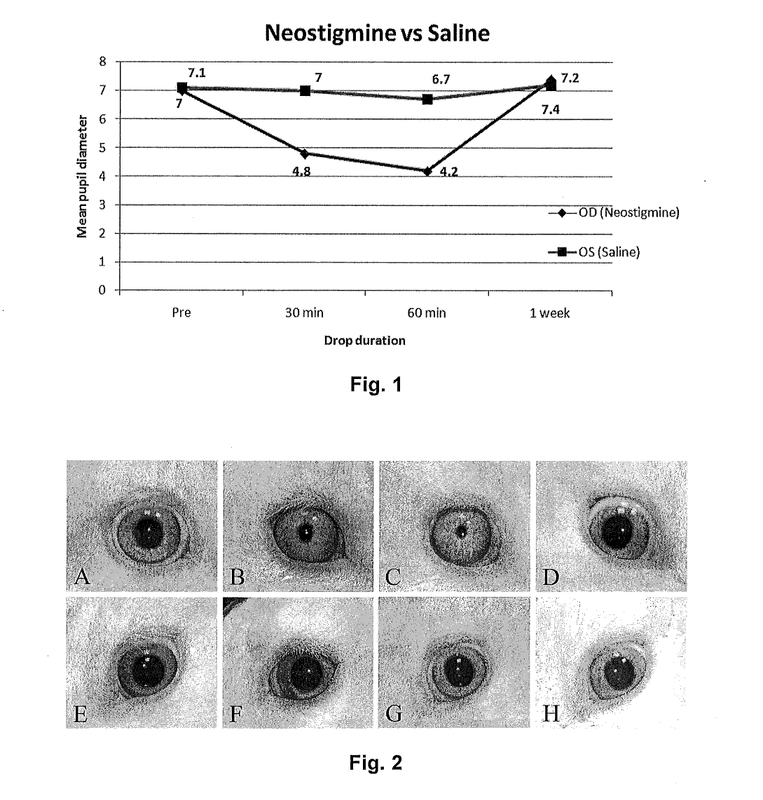

At baseline (FIG. 1) there was no significant difference in the mean [SD] pupillary size of the rabbits between the RE (7 mm [1.07]) and LE (7 mm [1.07], p=0.63). Significant miotic effect was observed in the RE compared to the LE at 30 and 60 minutes (respectively, 4.8 mm [1.86] vs 7.0 mm [1.09], p=0.0001; and 4.8 mm [1.86] vs 7.0 mm [1.09], p=0.0001). This significant miotic effect was also sustained at 90 minutes when comparing the RE to the LE (respectively, 3.2 [0.76] vs 7.0 [0.0], p=0.013). Administration of one drop of neostimine daily for 7 days caused no ocular inflammation. Measuring the pupillary size 24 hours after the last drop, revealed no significant difference between the RE and LE (respectively, 7.4 [0.94] vs 7.2 [1.01], p=0.062). A representative photograph of these pupillary changes is shown in FIG. 2.

### Patients

Six patients (3 males and 3 females) who presented with ptosis and/or ocular motility disturbance were enrolled in the study. Their ages ranged between 4.5 and 55 years (median=42 years, mean=36.4 years). The duration of their symptoms ranged between 1 and 18 months (median=4 months, mean=5.8 months).Three of them were not suspected before or investigated for MG, and hence received no treatment (Patients 1, 2 and 5). The other 3 (Paients 3, 4 and 6) were seropositive for AChR-antibodies, and one (Patient 6) had, as well, significant decrement (>10%) on repetitive nerve stimulation (RNS). Patient 3 had prednisone and pyridostigmine for 3 months, another (Patients 4) had prednisone for 2 months, and a third (Patient 6) had pyridostigmine for one year. Patient 3 received 30 mg of pyridostigmine 2 hours prior to the test whereas Patient 6 had 60 mg of pyridostigmine 8 hours earlier. The 6 patients manifested clinically with ocular MG, with no limb, axial muscles, oropharyngeal or respiratory muscle involvement.

All 6 patients with MG had an observable increase of the palpebral fissure height (documented by photography) of at least 2 mm, 30 minutes after neostigmine instillation. The response was dramatic in the 3 patients with no prior treatment for MG (Patients 1, 2 and 5).

Having described this invention above, it will be understood to those of ordinary skill in the art that the same can be performed within a wide and equivalent range of conditions, formulations and other parameters without affecting the scope of the invention or any embodiment thereof. Any document incorporated herein by reference is only done so to the extent of its technical disclosure and to the extent it is consistent with the present application and the disclosure provided herein. Deference is to be given to definitions, descriptions, wording, language, data, statements, etc. provided in the present document where any material disclosed in such incorporated references, to include definitions, descriptions, data and statements, conflicts with material provided in the present application.

We claim:

1. A method of diagnosing myasthenia gravis in a subject suspected of having myasthenia gravis, comprising administering neostigmine in a ptotic eye of a patient in an amount effective to increase palpebral fissure height in a patient having myasthenia gravis, and observing if palpebral fissure height prior to administration of the neostigmine is increased by the administration of the neostigmine, wherein an increase of 2 mm or more in palpebral fissure height is indicative of the presence of myasthenia gravis in the patient.

2. The method of claim 1, in which the neostigmine is one of neostigmine hydrochloride, neostigmine bromide or neostigmine methylsulphate.

3. The method of claim 1, in which the amount of neostigmine effective to increase palpebral fissure height in a patient having myasthenia gravis ranges from 0.01 mg to 1.0 mg.

4. The method of claim 1, in which the amount of neostigmine effective to increase palpebral fissure height in a patient having myasthenia gravis is 0.1 mg.

5. The method of claim 1, further comprising measuring palpebral fissure height from 30 to 60 minutes after administration of the neostigmine.

6. The method of claim 1, further comprising imaging the patient's ptotic eye prior to administration of the neostigmine and after administration of the neostigmine, determining the presence of an increase in palpebral fissure height after administration of the neostigmine by measuring palpebral fissure height in the images.

7. The method of claim 6 in which the imaging is photographing.

8. The method of claim 6, in which the neostigmine is neostigmine hydrochloride in 0.9% saline.

9. The method of claim 8 in which the amount of neostigmine effective to increase palpebral fissure height in a patient having myasthenia gravis is 0.1 mg and palpebral fissure height is measured 30-60 minutes after administration of the neostigmine.

10. The method of claim 1, in which the neostigmine is dispensed in a solution ranging from 0.5-5 mg/mL neostigmine.

11. A kit for use in determining the presence of myasthenia gravis in a patient comprising packaging and a controlled drop ophthalmic drug dispenser comprising an opthalmically acceptable solution of neostigmine.

12. The kit of claim 11, in which the neostigmine is neostigmine hydrochloride in 0.9% saline.

13. The kit of claim 11, in which the neostigmine is dispensed in a solution ranging from 0.5-5 mg/mL neostigmine.

14. The kit of claim 11, in which the controlled drop ophthalmic drug dispenser is a single-use dispenser.

15. The kit of claim 14, in which the kit comprises a plurality of single-use controlled drop ophthalmic drug dispensers.
